(19) United States
(12) Patent Application Publication
(10) Pub. No.: US 2003/0055018 A1
(43) Pub. Date: Mar. 20, 2003

(54) Modulation of phosphoinositide 3-kinase activity

(76) Inventors: Tung O. Chan, Philadelphia, PA (US); Philip N. Tsichlis, Willow Grove, PA (US)

Correspondence Address: Louis M. Heidelberger, REEDSMITHLLP, 2500 One Liberty Place, 1650 Market Street, Philadelphia, PA, 19103, US

(21) Appl. No.: 10/193,934

(22) Filed: Jul. 11, 2002

Related U.S. Application Data
(60) Provisional application No. 60/304,498, filed on Jul. 11, 2001.
(60) Provisional application No. 60/308,654, filed on Jul. 30, 2001.
(60) Provisional application No. 60/363,078, filed on Mar. 11, 2002.

(51) Int. Cl. A61K 48/00

(52) U.S. Cl. 514/44

(57) ABSTRACT

The present invention discloses methods of modulating phosphoinositide 3-kinase (PI3K) activity or phosphatidylinositol-3,4,5-P₃ (PIP3) production by targeting GTPase responsive domain or by expressing a p85 inhibitory domain of p85 subunit of phosphoinositide 3-kinase (PI3K) in cells. Methods for selecting modulators of PIP3 induction by small GTPases are also disclosed.

This application claims the benefit of U.S. Provisional Application No. 60/304,498 filed Jul. 11, 2001, U.S. Provisional Application No. 60/308,654, filed Jul. 30, 2001 and U.S. Provisional Application No. 60/363,078, filed Mar. 11, 2002 (each of which applications are hereby incorporated by reference in their entirety).

Pursuant to 35 U.S.C. §202 it is acknowledged that the U.S. Government has certain rights in the invention described herein, which was made in part with funds from the Public Health Service, Grant Number R01CA57436.

### FIELD OF THE INVENTION

The present invention relates to modulation of phosphoinositide 3-kinase (PI3K) activity or phosphatidylinositol-3,4,5-P₃ (PIP3) production in mammalian cells. Specifically, the present invention discloses specific domains within a p85 subunit of the PI3K and newly identified motifs within the domains as targets for manipulating PI3K activity. More specifically, the invention provides methods and compositions for modulating phosphoinositide 3-kinase (PI3K) activity in mammalian cells by targeting different subunits of the PI3K. More specifically, the invention identifies for modulating PI3K activity.

### BACKGROUND OF THE INVENTION

Phosphoinositide 3-kinase (PI3K) type IA is a heterodimer of a regulatory subunit (p85) and a catalytic subunit (p110). PI3K catalyses the phosphorylation of PtdIns-4, 5-P₂ to form PtdIns-3, 4, 5-P₃ (Wymann et al., 1998, Biochim Biophys Acta 1436, 127-50). PtdIns-3, 4, 5-P₃ (PIP3) in turn regulates a diverse array of cellular functions including apoptosis, proliferation, differentiation, and intermediary metabolism (Chan et al.,1999, Ann Rev Biochem 68, 965-1015; Rameh et al., 1999, J Biol Chem 274, 8347-50). The cellular levels of phosphatidylinositol-3,4,5-P₃ are elevated in almost all cancers through the acquisition of mutations in tyrosine kinases, small GTPases and the phosphatidylinositol-3 phosphatase (PTEN).

PI3K is activated rapidly following growth factor stimulation and cross-linking of cell adhesion molecules (Rameh et al., 1999, J Biol Chem 274, 8347-50). Earlier studies suggested that the binding of the regulatory subunit of PI3K to tyrosine-phosphorylated molecules is directly responsible for PI3K activation (Backer et al., 1992, Embo J 11, 3469-79; Carpenter et al., 1993, J Biol Chem 268, 9478-83). However, recent in vitro studies (Layton et al., 1998, J Biol Chem 273, 33379-85) and earlier in vivo studies (Fukui et al., 1989, Mol Cell Biol 9, 1651-8; Varticovski et al., 1991, Mol Cell Biol 11, 1107-13) suggested that binding is not sufficient to activate PI3K. Other studies showed that PI3K is also activated by small GTPase molecules such as Ras and Rac1 (Nishida et al., 1999, Oncogene 18, 407-15; Rodriguez-Viciana et al., 1994, Nature 370, 527-32). These molecules activate PI3K by diverse, although not clearly defined, mechanisms. Thus, PI3K activation by Ras has been correlated with direct binding between Ras and p110 (Rodriguez-Viciana et al., 1994, Nature 370, 527-32), while PI3K activation by Rac1 may be mediated by binding of Rac1 to p85 (Tolias et al., 1995, J Biol Chem 270, 17656-9; Zheng et al., 1994, J Biol Chem 269, 18727-30). Moreover, constitutively-active Ras activates the PI3K/Akt pathway in synergy with Src (Datta et al., 1996, J Biol Chem 271, 30835-9).

Despite extensive studies on the mechanism of PI3K activation by small GTPases and tyrosine kinases, how these molecules transduce signals for PI3K activation in cells is not known. Given the significance of PI3K pathway, PIP₃ and its target Akt, in human cancer and other disorders, it would desirable to modulate PI3K activity in cells. Methods of modulating PI3K activity would be feasible if the precise sites on PI3K targeted by small GTPases and tyrosine kinases, or how these molecules regulate PI3K are known.

### SUMMARY OF THE INVENTION

In the present invention, it has been found that the p85 subunit of PI3K contains a molecular switch that regulates PI3K in response to signals transduced by tyrosine kinases and small GTPases.

This molecular switch has two domains; GTPase-Responsive Domain (GRD) and an inhibitory domain. Small GTPases (e.g., H-Ras, Rac1) activate PI3K by targeting the GTPase-Responsive Domain. The stimulatory effect of these molecules, however, is blocked by the inhibitory domain, which functions by binding to tyrosine phosphorylated molecules and is neutralized by tyrosine phosphorylation. The complementary effects of tyrosine kinases and small GTPases on the p85 molecular switch result in synergy between these two classes of molecules toward the activation of PI3K (and hence Akt) pathway.

Accordingly, in one aspect of the invention, a method of of modulating phosphoinositide 3-kinase (PI3K) activity or phosphatidylinositol-3,4,5-P₃ (PIP3) production by targeting GTPase responsive domain (GRD) or by expressing a p85 inhibitory domain of p85 subunit of phosphoinositide 3-kinase (PI3K) in cells is provided.

In another aspect of the invention, a method of modulating ras-induced phosphatidylinositol-3,4,5-P₃ (PIP3) synthesis in a mammalian cell is provided. This method involves administering a vector capable of expressing a p85 inhibitory domain comprising amino acids set forth in FIG. 14B to a mammal. The vector is targeted to a mammalian cell such as a cancer cell (prostate, breast or lung cancer cell). Tissue specific delivery of the p85 inhibitory domain can be achieved by using techniques such as tissue specific promoters or by using vectors with modified tropisms.

In still another aspect of the invention, a recombinant polynucleotide segment having a polynucleotide fragment of phosphoinositide 3-kinase (PI3K) is provided. The segment does not encode GRD of the p85. The polypeptide may have a mutation (for example Y688F) to disable neutralization of the polypeptide by tyrosine kinase transmitted signals.

### BRIEF DESCRIPTION OF THE DRAWINGS

FIG. 1 shows a p85 inhibits while p85Δonc enhances PI3K activation by small GTPases). (A) Schematic diagrams and expression of p85-WT and p85Δonc constructs. (B) Upper panel. H-Ras-induced PtdIns-3, 4, 5-P₃ production. (B) Lower panel. H-Ras induced Akt phosphorylation. (C) PtdIns-3, 4, 5-P₃ induction is inhibited by the PI3K inhibitor, wortmannin. (D) p85Δonc synergizes with Rac1 to induce PtdIns-3, 4, 5-P₃ synthesis and Akt activation.

FIG. 2 shows that Membrane-targeted p85 continues to block PI3K/Akt activation by H-Ras. (A) Schematic diagrams of HA-tagged Myrp85-WT and Myrp85Δonc. (B) Myrp85-WT inhibits PI3K activation by H-Ras. (C) Myrp85Δonc enhances PI3K activation by H-Ras.

FIG. 3 shows that the carboxy-terminally truncated inter-SH2 domain of p85 synergizes with Ras and Rac1 to activate the PI3K/Akt pathway. (A) Schematic diagram of wild-type p85 and sequence comparison of inter-SH2 domains. (B) The p85-iSH2 domain lacking 52 amino acids from its carboxy terminus synergizes with both activated H-Ras and activated Rac1. (C) The p85 IKR motif is a required component of the GRD domain. Schematic diagrams on the left of (B) and (C) show the domain composition of transfected constructs.

FIG. 4 shows that the carboxy terminal p85 inhibitory domain is modular and consists of the LED and cSH2 motifs. (A) Schematic diagrams of p85 constructs. (B) p85-CT and p85DN inhibit the activation of the PI3K/Akt pathway by H-Ras-G12V. (C) The PI3K inhibitory domain consists of the LED and cSH2 motifs. (D) The cSH2 and IKR/LED motifs alone have no inhibitory activity.

FIG. 5 shows that the PI3K inhibitory domain inhibits p110α, but not p110γ. The inhibition is independent of PTEN. (A, B) p85-CT and p85-DN block Akt activation by constitutively-active p110α-CAAX. (C) p85-CT and p85-DN block Akt activation by Ras in the absence of PTEN.

FIG. 6 shows that Ras-independent Src signals neutralize the p85 inhibitory domain. (A) Schematic diagram of the chicken Src tyrosine kinase and its mutants used in this report. (B) p85-WT inhibits Akt activation by Ras, but not by Src. (C) Mutation of the Src myristoylation signal does not affect Src tyrosine kinase activity, but abolishes Src-induced activation of the Ras/ERK pathway and the PI3K/Akt pathway. (D) The non-myristoylated Src mutant synergizes with H-Ras to activate the PI3K/Akt pathway. (E) The non-myristoylated Src abolishes the inhibition of PtdIns-3, 4, 5-P₃ production by p85.

FIG. 7 shows that Mutations of the phosphotyrosine binding site and the Tyr688 phosphorylation site of the cSH2 motif neutralize the inhibitory domain. (A) Schematic diagram of p85-CT. (B) The R649L and Y688D mutants of p85-CT do not inhibit activation of the PI3K/Akt pathway by Ras. (C) The phosphorylation site mutant Y688F continues to inhibit PI3K/Akt activation by Ras. (D) p85-CT blocks cell transformation by activated H-Ras.

FIG. 8 shows that complimentary roles of tyrosine kinase and small GTPase signals induced by integrin or growth factor stimulation, in PI3K/Akt activation and cell survival. (A) Inhibition of fibronectin-stimulated Akt activity by kinase-inactive Src. (B) Activated Ras and EGF cooperate to support long-term cell survival (10 days) and PI3K/Akt activation in HaCaT keratinocytes in suspension cultures. (C) A model of PI3K activation by tyrosine kinase- and small GTPase signals.

FIG. 9 shows that Activation of the PI3K/Akt pathway by Ras and Rac1 does not depend on the direct interaction between PI3K and these molecules. (A) Myrp85Δonc activates the PI3K/Akt pathway in synergy with H-Ras-G12V/E37G, a mutant that does not bind p110. (B) Ras-binding-domain (RBD) of p110 does not inhibit H-Ras-G12V-induced Akt activation. (C) BCR domain of p85 does not inhibit Rac1 Q61L-induced Akt activation.

FIG. 10 shows that Phosphotyrosine peptides enhance PtdIns-3-P, not PtdIns-3,4,5-P₃ synthesis by p85 immunoprecipitates in vitro. (A) In vitro PI3K assays NIH3T3 using serum-starved. (B) Graphs showing the synthesized ³²P-labeled PtdIns-3-P and PtdIns-3, 4, 5-P₃ were measured by Phosphorimager.

FIG. 11 shows that Ras and p85Δonc do not activate Akt in PDK1(−/−) embryonic stem cells.

FIG. 12 shows that the Src catalytic domain and other tyrosine kinases, Syk, Abl, PDGF-R and IGF-1-R, but not Fak, synergize with Ras to activate the PI3K/Akt pathway. (A) The Src catalytic domain, NMSrc-Kin, and Syk-Y130E synergize with Ras to activate Akt. (B) Non-myristylated Abl (NMAb1) synergizes with Ras. (C) The catalytic domain of Fak (Fak-Kin) does not synergize with Ras. (D) Stimulation of the PDGF receptor synergizes with Ras. (E) The catalytic domain of the IGF-1 receptor (IGF1-R-Kin) synergizes with Ras.

FIG. 13 shows that Src and Abl synergize with small GTPases (H-Ras, R-Ras, Rac1 and CDC42), but not with p110, to activate the PI3K/Akt pathway. (A) Rac1 and CDC42, but not RhoA, synergize with non-myristoylated Abl tyrosine kinase in Akt activation. (B) Src synergizes with R-Ras to activate Akt. (C) Non-myristylated Src does not synergize with P110α-CAAX.

FIG. 14 shows amino acid sequence of human p85 alpha (A) GTPase-Responsive Domain (B) Inhibitory Domain.

FIG. 15 shows amino acid sequence of bovine p85 alpha (A) GTPase Response Domain (B) Inhibitory Domain.

### DETAILED DESCRIPTION OF THE INVENTION

The present invention relates to the regulation of Phosphoinositide 3-kinase (PI3K)/Akt pathway in cells and the molecules for regulating the pathway. The inventon discloses that p85, a regulatory subunit of PI3K, contains GTPase-Responsive Domain and an inhibitory domain which together form a molecular switch that regulates PI3K. A novel regulatory mechanism disclosed herein explains how tyrosine kinases (cytoplasmic and receptor) and small GTPases regulate PtdIns-3, 4, 5-P₃ production (PIP3) by type IA PI3K. The invention also discloses how the carboxy-terminally truncated p85 functions as an oncogene. From this disclosure, it is now known that the PI3K activity is under the dual control of a p85 inhibitory domain which is regulated by binding to tyrosine phosphorylated molecules and by tyrosine phosphorylation, and a GTPase-Responsive Domain (GRD). Because of the inhibitory domain, PI3K either does not respond or responds poorly to signals from small GTPases such as (e.g., Ras or Rac1). Removal of the inhibitory domain, as in the case of the p85 oncogene, or neutralization of its inhibitory function by tyrosine phosphorylation, allows small GTPases to activate PI3K through the GRD. The LED motif is required part of the inhibitory domain. Accordingly, the inhibitory domain and the GRD together, form a molecular switch that regulates the enzyme.

The presnt invention also provides methods of modulating phosphoinositide 3-kinase (PI3K) activity or phosphatidylinositol-3,4,5-P₃ (PIP3) production in mammalian cells. This is accomplished by interfering with GTPase-Responsive Domain in order to modulate or control PI3K activity in mammalian cells. For example, inactivating GTPase-Responsive Domain, such as by specific mutations so that this domain does not repond to siganls transduced by small GTPases. Preferably, the GTPase-Responsive Domain is abrogated so that it does not repond to siganls transduced by Ras. The GTPase-Responsive Domain of humans and bovine p85 alpha subunit of PI3K spans from amino acids 490 to 575. Preferably, amino residues in IKR motif (550-575) should be the targets of mutations. Preferred amino acids for mutations are lysine at position 567, Leucine at position 570 and/or Leucine at position 573 of the PI3K type 1A sequence. For example, these amino acids can be replaced with alanine (i.e., K567-L570-L573 to A). Other preferred mutations are arginine at position 574, lysine at 575 and/or arginine at position 577. These amino acids may also be replaced with alanine (i.e., R574-K575-R577 to A). These mutations modulate GRD function.

Another way of disabling the GTPase-Responsive Domain is by way of antisense interference. Antisense technology is well known to one skilled in the art. Based on the publicly available human p85-alpha nucleic acid sequence, one skilled in the art can make an antisense molecule targeted to a nucleic acid portion, for example, encoding amino acids 550 to 575.

According to another aspect of the invention, Ras-induced phosphatidylinositol-3,4,5-P₃ (PIP3) synthesis in a mammalian cells can be modulated. It can be carried out by delivering a vector capable of expressing a p85 inhibitory domain to a mammal, preferably a human. The vector can be delivered by direct injection. Alternatively, the vector can be so designed that it can be targed to a specific tissue by delivering systemically. One skilled in the art would know how to accomplish tissue specific delivery the recombinant p85 inhibitory domain because systemically deliverable vectors for tissue specific therapeutic gene expression have been reported. Some of these vector designs were based on adenovirus and retrovirus based vectors. These designs include vectors with tissue specific promoters or modified tropisms. See, Greogry et al., Gene Therapy Using Replication Competent Targeted Adenoviral Vectors, WO 96/34969; Hallenbeck et al., Vectors for Tissue-Specific Replication, WO 96/17053, Curiel, Targeted Adenovirus Vectors, WO 97/20575; Curiel, Targeted Adenovirus Vectors, U.S. Pat. No. 5,871,727; Sosnowski et al., Adenoviral Vectors with Modified Tropism, WO 9840508. In addition, the genes delivered systemically by retroviral vectors are integrated and expressed only in dividing cells (e.g., cancer cells) because of the ability of the virus to integrate into the host cell genome during cell division

Methods for selecting (or screening for) modulators of PI3K activity or phosphatidylinositol-3,4,5-P₃ (PIP3) induction by a small GTPase are also provided herein. Modulation, as used herein, refers to a change or an alteration in the PI3K activity or phosphatidylinositol-3,4,5-P₃ (PIP3) induction. Modulation may be an increase or a decrease in protein activity, a change in binding characteristics, or any other change in the biological or functional properties of PI3K.

Screening for modulators (inhibitors or enhancers) can be done in host cells and it may involve highthroughput techniques. A host cell (test cell) of a given type transfected with a GRD construct to express GRD of p85 subunit of PI3K in the cell, and the cells expreesinng the small GTPase can be used for screening. The small GTPases must be activated GTPases and capable functionally interacting with the GRD. The small GTPases include endogenous small GTPases activated either by mutations or by upstream regulators of small GTPases (grow factors, cytokines) and/or exogenously introduced activated small GTPase (e.g., a vector construct expressing a small GTPase). As a control, a host cell of the same type without the GRD construct is maintained. The control host cell may be transfected with a small GTPase expressing construct if it is done with the test cell. The host cells of a given type can be for example, BaF3 cells, U87MG (PTEN-null) glioblastoma cells, epithelial cells etc., which are well known to one skilled in the art. Preferably any cell that can be genetically induced so as to cause changes in the growth phenotype so that they continue to divide (immortalization). Tumor or cancer cells continue to divide under circumstances in which their cellular counterparts do not. Prior art known cancer cells can be used as host cells. The host cells are then contacted with a compound to be tested or screened. The compound can be an organic or inorganic molecule, including but not limited to proteins, peptides, polysaccharides, lipids, nucleic acids, small organic molecules, inorganic compounds, and derivatives thereof that are cell permeable. Some examples of small molecules are acids (for example acetic acid, salicylic acid, ascorbic acid) bases, formamide, amino acids and their derivatives (for example protoheme, cytochrome heme) inorganic molecules (for example phosphoric acid), acetycholine, sugars, prosthetic groups, cofactors and inhibitors (for example, Flavin adenine dinucleotide, riboflavin, NAD, NDP⁺, NADPH, folic acid, methotrexate) aspirin, palmitic acid, caffeine, beta-mercaptoethanol, urea, minerals or vitamins. The host cells are contacted with a test compound for sufficient time so that the compound is taken up by the cell. The test compound is applied to the cells either before transfections or after transfections or simultaneously with the transfections. Screening for drugs using live cells and reagents are routine in the art and can be optimized. After exposing the host cells to a test compound for sufficient time, cells are analyzed for PIP3 levels or PI3K activity. Any differences between the test and the control cells can be attributed to modulation by the test compound.

A molecule or a compound which, when bound to GRD, enhances the biological activity of PI3K (and hence PIP3 levlels) is an agonist. Agonist as used herein is that compound or molecule which, when bound to GRD, causes a change in GRD which regulates PI3K. Agonists may include proteins, nucleic acids, carbohydrates, or any other molecules which bind to GRD A molecule or a compound which, when bound to GRD, inhibits the biological activity of PI3K is an antagonist or inhibitor. These may include proteins, nucleic acids, carbohydrates, or any other molecules which bind to GRD.

In a further aspect of the invention, recombinant molecules containing the p85 inhibitory domain is provided. Because the p85 inhibitory domain is found in the present invention to disrupt PI 3-kinase activity and because p85 has been shown to block the activated Ras-induced cell transformation in NIH3T3 cells, these molecules can be used, for example, as anti-neoplastic agents. This effect is linked to blocking Ras-induced PIP₃ synthesis but not MAP kinase activity. By contrast, Ras inhibitors, notably farnesyltransferase inhibitors, prevent multiple events downstream of Ras activation. In addition, these inhibitors are not specific to Ras because they block farnesylation and, thus, membrane transport of many other molecules. In fact, the clinical efficacy of famesyltransferase inhibitors may be related to their effects on RhoB but not Ras. Furthermore, the p85 inhibitory domain specifically inhibits type IA PI 3-kinase without significantly affecting the closely related type IB class. By contrast, the widely used PI-3-kinase inhibitors wortmannin and LY294002 lack specificity as they inhibit not only type IA PI 3-kinase but all known PI 3-kinase subtypes.

The p85 inhibitory domain, composed of 135 amino acids, potently inhibits Ras-induced PIP₃ synthesis by neutralizing the Ras/Rac1 small GTPase-Responsive Domain. Neutralization may be caused by interactions of the IKR region within GTPase-Responsive Domain with the LED region of the inhibitory domain. Either the full-length p85 inhibitory domain or fragments thereof may be used. For example, fragments may be generated by N-terminal, C-terminal and internal deletions of the inhibitory domain using a construct that expresses the inhibitory domain fused to green-fluorescence protein. The activity of the deleted constructs can be determined using the assay that measures the blocking of Ras-induced PIP₃ synthesis in NIH3T3 cells. Preferably, inhibitory peptides shorter than 60 amino acids may be generated and used. The inhibitory activity of these peptides can be tested by introducing them into cells, for exmaple, by HIV TAT-mediated protein transduction or by transfection, using commercially available transfection reagents (ActiveMotif).

The effectiveness of the p85 inhibitory domain and its peptide derivatives can be tested against cell-transformation and tumorigenesis by oncogenic mutations that target the PI 3-kinase/PTEN/Akt pathway. These include the activation G12Vmutation of H-Ras, the fusion of BCR with the ABL tyrosine kinase and the inactivation of the tumor suppressor gene, PTEN. To determine the ability of the p85 inhibitory domain or fragments thereof to block the biological effects of these oncogenic mutations, any of a number of suitable assays can be used. The following assays may be used:

Anchorage independent survival of EGF-stimulated epithelial HaCaT cells expressing Ras. This assay, which measures survival of epithelial cells when grown in suspension, permits the identification of factors and signaling pathways that support anchorage-independent survival of non-tumorigenic epithelial HaCaT cells. In this system, activated Ras supports long-term survival of HaCaT cells (>10 days) in synergy with epidermal growth factor (EGF). More important, survival mirrors the synergistic induction of PIP₃ synthesis by Ras and tyrosine kinases activated by EGF. Using this assay system, one can measure the activities of the p85 inhibitory domain and its peptide derivatives.

BCR/ABL induced IL-3 independence of Ba/F3 cells. BCR/ABL-induced PIP₃ synthesis and the activation of its target Akt/PKB, are required for hematopoictic cell transformation. Transformation of the murine proB cell line, Ba/F3, by BCRIABL via the PIP₃/Akt pathway is characterized by the ability of the transformed cells to grow in the absence of IL3. BCR/ABLtransformed Ba/F3 cells can be engineered to express the full-length inhibitory domain or its partial deleted derivatives. This will allow one to determine the effects of these constructs to block PIP₃ induction and cell transformation by BCR/ABL. Alternatively, the smallest peptide derivatives of the inhibitory domain can be tested directly for their inhibitory activity as described above.

Anchorage independent survival and oncogenic potential of U87MG (PTEN-null) glioblastoma cells. One of the most frequently mutated tumor suppressor genes in cancer is PTEN which suppresses tumorigenesis by reducing the cellular concentration of PIP₃. Since enforced expression of either PTEN or the p85 inhibitory domain block PIP₃ production, overexpression of p85 inhibitory domain in cells that lack PTEN function may significantly reduce PIP₃ level.

Some of the well known in vivo models can also be used including, but not limited to,: BCR/ABL-induced chronic myeloid leukemia (CML) and B-cell acute lymphoblastic leukemia (ALL) which can be both induced by retroviral bone marrow transduction/transplantation in mice. Tumors induced in MMTV-Ras transgenic and PTEN heterozygous (Pten^{+/-}) mice.

The molecular switch located in p85 regulatory subunit composed of an activation domain and an inhibitory domain, complementary roles of small GTPases and tyrosine kinases in regulating PI 3K through this switch and the p85 inhibitory domain as a molecule to down regulate PIP3 are described in greater detail in the form of specific working examples.

### EXAMPLES

The following examples further illustrate the present invention, but of course should not be constructed as in any way limiting its scope. In other words, the examples are illustrative, but do not limit the invention. The examples below are carried out using standard techniques and procedures that are well known and routine to those of skill in the art. A description of the procedures used is also provided following the examples. All animal methods of treatment or prevention described herein are preferably applied to mammalian cells (both isolate cells and cells in vivo), most preferably to humans.

### Example 1

### Wild-type p85 and Carboxy-Terminally Truncated p85 Exert Opposite Effects on PtdIns-3, 4, 5-P₃ Synthesis Induced by H-Ras or Rac1

The carboxy-terminal SH2 domain and 52 adjacent amino acids from the inter-SH2 (iSH2) region is referred to herein as p85Δonc.

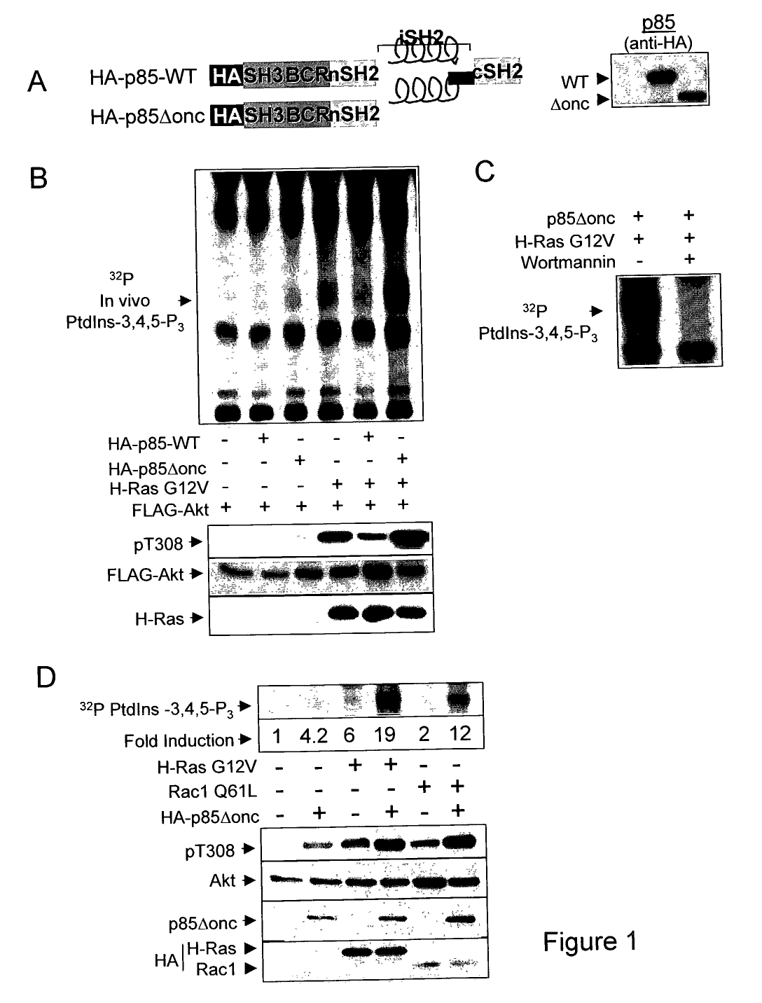

In FIG. 1, it is shown that p85 inhibits while p85Δonc enhances PI3K activation by small GTPases. (A) Schematic diagrams and expression of transiently transfected HA-tagged p85-WT and p85Δonc constructs. (B) Upper panel. p85-WT inhibits while p85Δonc enhances HRas-induced PtdIns-3, 4, 5-P₃ production. The indicated constructs were transiently transfected into NIH3T3 cells. Sixteen hours after transfection, the cells were cultured in serum-free media. Five hours later, they were labeled with ³²P-orthophosphate for 1.5 hours. Total phospholipids were analyzed by thin layer chromatography. (B) Lower panel. p85-WT inhibits while p85Δonc enhances H-Ras induced Akt phosphorylation. Immunoblots of cell extracts from duplicate transfections were probed with an antibody that recognizes Akt phosphorylated at Thr308 or with antibodies that recognize the proteins expressed from the transfected constructs. (C) PtdIns-3, 4, 5-P₃ induction by p85Δonc and constitutively-active H-Ras is inhibited by the PI3K inhibitor, wortmannin. NIH3T3 cells were transiently transfected with the indicated constructs and they were labeled with ³²P-orthophosphate as described in B. 45 min prior to lipid extraction, the cells were treated with 100 nM wortmannin or DMSO. (D) p85Δonc synergizes with Rac1 to induce PtdIns-3, 4, 5-P₃ synthesis and Akt activation. NIH3T3 cells were transiently transfected with the indicated constructs. ³²P-labeled PtdIns-3, 4, 5-P₃ was measured as described in B. Akt phosphorylation at Thr308 and expression of transfected constructs were determined by probing immunoblots with the corresponding antibodies.

Specifically, to address the question whether p85Δonc and p85 differ in their ability to regulate PI3K activation by H-Ras., a thin layer chromatography-based assay was used (Maehama et al., 1998, J Biol Chem 273, 13375-8) to separate PtdIns-3, 4, 5-P₃ from lipid extracts of ³²P-labeled NIH3T3 cells transfected with Hemagglutinin (HA)-tagged constructs of wild-type p85 (HA-p85-WT) or p85Δonc (HA-p85Δonc), and constitutively-active H-Ras (H-Ras G12V) in the combinations shown in FIG. 1B. HA-p85-WT and HA-p85Δonc were both expressed at similar levels (FIG. 1A). Consistent with the findings of Jimenez et al.(Jimenez et al., 1998, Embo J 17, 743-53), HA-p85Δonc alone, weakly activated PI3K in vivo (FIG. 1B, upper panel). The p85 and p85Δonc, however, had diametrically opposite effects when cotransfected with Ras in that whereas p85 inhibited the activation of PI3K, p85Δonc enhanced it dramatically. FIG. 1C shows that the synergy between H-Ras and p85Δonc is wortmanninsensitive, and therefore, PI3K-dependent.

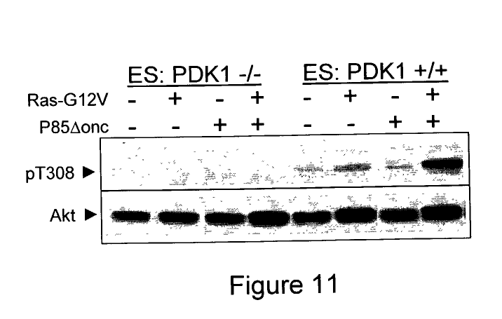

PtdIns-3, 4, 5-P₃ synthesized by the activated PI3K is required for activation of the Akt kinase (Chan et al., 1999, Ann Rev Biochem 68, 965-1015). As shown here, the Akt phosphorylation and activity correlate with the intracellular levels of PtdIns-3, 4, 5-P₃. An immunoblot of cell lysates derived from the cells in the preceding experiment was probed with an antibody that recognizes the phosphorylated Thr308 motif of Akt. The results (FIG. 1B, lower panel) show correlation between Akt phosphorylation and PtdIns-3, 4, 5-P₃ levels. The fact that Ras and p85Δonc activate Akt via PI3K and not via direct phosphorylation of Akt was further confirmed by experiments showing that the combination of Ras and p85Δonc does not activate Akt in PDK1 (−/−) cells (see FIG. 11).

It is also shown here that p85Δonc synergizes with small GTPases other than Ras to activate the PI3K pathway. FIG. 1D, upper panel, shows that the combination of transiently transfected, constitutively-active Rac1 (Rac1 Q61L) and p85Δonc in NIH3T3 cells dramatically enhanced the intracellular levels of PtdIns-3, 4, 5-P₃. The lower panel of the same figure shows again that the levels of PtdIns-3, 4, 5-P₃ correlate with the phosphorylation of Akt at Thr308. The in vitro kinase activity of Akt to monitor the in vivo activity of PI3K was used. By direct measurement of the intracellular levels of PtdIns-3, 4, 5-P₃.

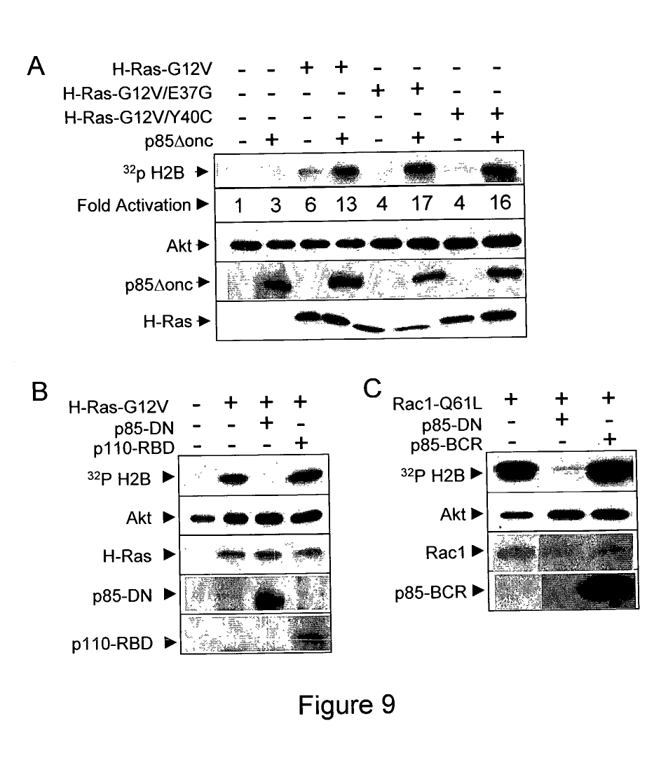

PI3K activation via the synergistic actions of Rac1 and p85Δonc does not depend on the binding of Rac1 to p110, because these proteins do not interact (Tolias et al., 1995, J Biol Chem 270, 17656-9; Zheng et al., 1994, J Biol Chem 269, 18727-30). Similarly, it was reported that both H-Ras G12V/E37G and H-Ras G12V/Y40C, two activated H-Ras mutants of which only the latter interacts with p110 (Rodriguez-Viciana et al., 1997, Cell 89, 457-67; White et al., 1995, Cell 80, 533-41), synergize equally well with p85Δonc to activate PI3K (see FIG. 9). The same figure shows that over-expression of the BCR domain of p85, which is known to bind Rac1 (Tolias et al., 1995, J Biol Chem 270, 17656-9; Zheng et al., 1994, J Biol Chem 269, 18727-30), does not inhibit PI3K activation by Rac1. Therefore the Rac1/p85 interaction is also not required for PI3K activation.

### Example 2

### Membrane Localization of p85 is not Sufficient to Activate PI3K

Truncated p85 is thought to activate PI3K by directing it to the plasma membrane (Jimenez et al., 1998, Embo J 17, 743-53).

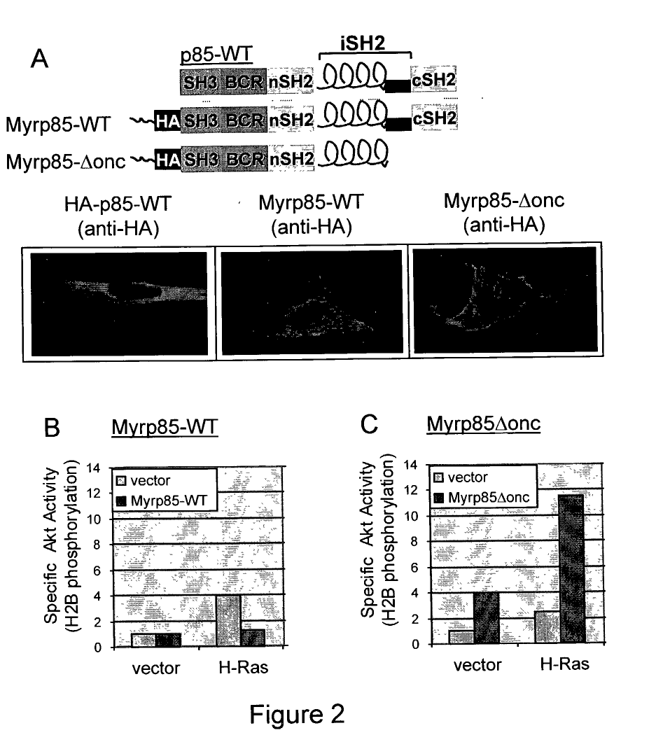

In FIG. 2, it is shown that membrane-targeted p85 continues to block PI3K/Akt activation by H-Ras. (A) Schematic diagrams of HA-tagged Myrp85-WT and Myrp85Δonc. NIH3T3 cells transfected with these constructs were fixed with paraformaldehyde and they were incubated with the anti-HA antibody. Bound antibody was visualized by fluorescein isothiocyanate (FITC)-labeled goat anti-mouse antiserum and fluorescence confocal microscopy. (B) Myrp85-WT inhibits PI3K activation by H-Ras. Constitutively-active H-Ras and FLAG-cAkt were transfected into NIH3T3 cells in combination with Myrp85-WT or vector. The in vitro phosphorylation of Histone 2B (H2B) by FLAG-Akt immunoprecipitates was measured by phosphorimager. Akt levels in the immunoprecipitates were measured by immunoblotting and laser densitometry scanning (Molecular Dynamics). The bar graphs show the specific activity of Akt as calculated from these two values. H2B phosphorylation is presented in arbitrary units. (C) Myrp85Δonc enhances PI3K activation by H-Ras. Constitutively-active H-Ras was transfected in combination with Myrp85Δonc or vector into NIH3T3 cells. The specific activity of Akt was determined as in B.

Specifically, to show whether membrane-associated full-length p85 is functionally similar to the carboxy-terminally truncated protein the p85 fused at its amino-terminus to a peptide encoding the Src myristoylation signal (Myrp85-WT) (FIG. 2A), was transiently expressed in NIH3T3 cells in combination with FLAG-Akt or FLAG-Akt plus H-Ras. Myrp85Δonc was used as a control. The cells were lysed forty-eight hours later, following overnight serum starvation. Akt activation in this, and subsequent experiments, was determined by measuring the phosphorylation of histone 2B (H2B) by immunoprecipitated Akt.

In vitro kinase assays of FLAG-Akt revealed that membrane-targeted p85 is similar to the wild-type protein in that it does not activate the PI3K/Akt pathway by itself and that it blocks activation of the pathway by H-Ras (FIG. 2B). Myrp85Δonc, on the other hand, was similar to p85Δonc in that it activated the PI3K/Akt pathway both alone and in synergy with H-Ras (FIG. 2C). Thus, association of p85 with the plasma membrane is not sufficient to activate PI3K. Therefore, the carboxyl terminal truncation of p85 is unlikely to activate PI3K by targeting the enzyme to the plasma membrane. The truncation unveils a domain in p85 that responds to Ras- or Rac1-transduced signals to activate PI3K (GTPase-Responsive Domain, GRD).

### Example 3

### A protein Motif Within the iSH2 Domain of p85 Synergizes with H-Ras and Rac1 to Activate the PI3K/Akt Pathway

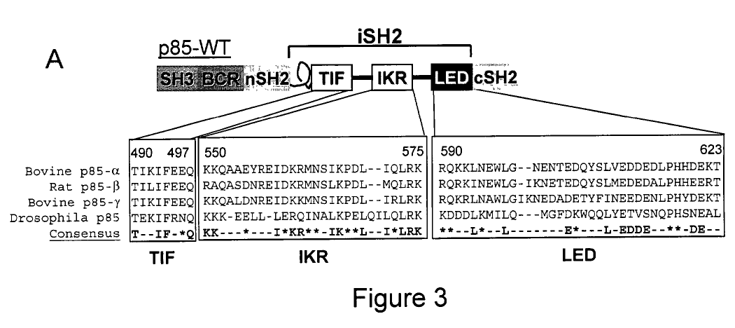

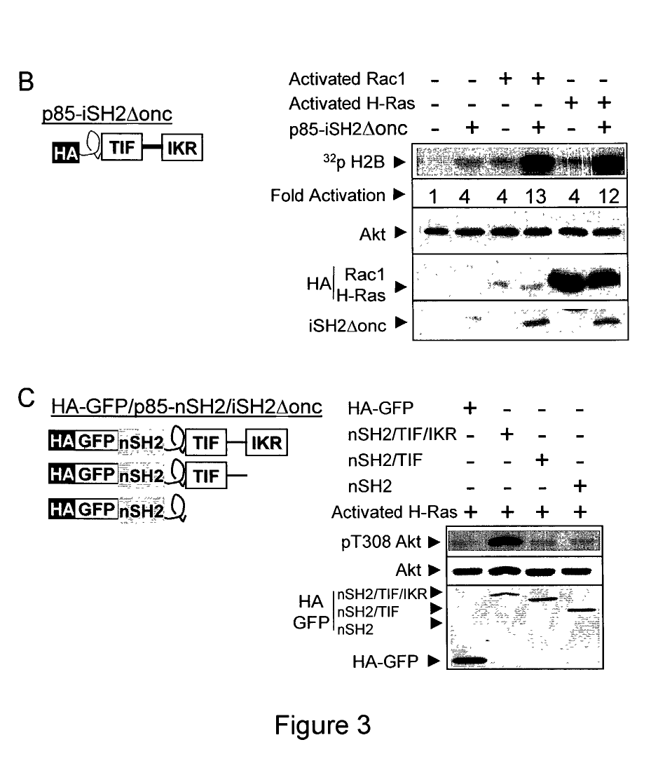

In FIG. 3, it is shown that The carboxy-terminally truncated inter-SH2 domain of p85 synergizes with Ras and Rac1 to activate the PI3K/Akt pathway. (A) Schematic diagram of wildtype p85 and sequence comparison of inter-SH2 domains. Line-up of the amino acid sequence of several p85 iSH2 domains, bovine p85-α(M61745), Rat p85-β(NM_{—}022185), bovine p85-γ (AF036256) and Drosophila p85(Y11143). Conserved amino acid motifs include: TIF (p110 binding motif), IKR and LED. (B) The p85-iSH2 domain lacking 52 amino acids from its carboxy terminus synergizes with both activated H-Ras and activated Rac1. Left panel. Schematic diagram shows an HA-tagged construct of the inter-SH2 domain carrying the same carboxy-terminal truncation as p85Δonc (p85-iSH2Δonc). Right panel. The indicated constructs were transiently transfected into NIH3T3 cells. Akt activity was measured by in vitro phosphorylation of H2B. Expression of proteins encoded by the transfected constructs was determined by probing immunoblots of total lysates with the anti-HA antibody. (C) The p85 IKR motif is a required component of the GRD domain. Schematic diagram on the left shows the domain composition of transfected constructs. These constructs were transfected into NIH3T3 cells together with an expression construct of constitutively active H-Ras. Akt activity was measured by Akt phosphorylation at Thr308. Expression of the transfected constructs were determined by probing immunoblots with the corresponding antibodies.

The p85 regulatory subunit of PI3K contains several protein domains, including SH3, BCR homology, two SH2 domains and an inter-SH2 domain (iSH2) (FIG. 3A). Computer modeling of the iSH2 domain suggested that this region resembles a coiled-coil structure (Wymann et al., 1998, Biochim Biophys Acta 1436, 127-50). A motif (TIF), previously mapped within this structure, binds the PI3K catalytic subunit (p110) (Dhand et al., 1994, Embo J 13, 511-21). The TIF motif is highly conserved across p85 sub-types (alpha, beta and gamma) and across species (bovine, rat and drosophila) (FIG. 3A). Other conserved motifs include a motif, named IKR, which is rich in isoleucine, lysine and arginine residues and a motif, named LED, which is rich in leucine, glutamic acid and aspartic acid residues. The LED motif is located downstream from the IKR motif and corresponds to the carboxy-terminus of the iSH2 domain. The carboxy-terminus of the IKR motif corresponds to the break-point of the p85 oncogenic truncation.

The p85 oncogenic truncation separates the TIF and IKR motifs from the LED motif. To show whether the TIF/IKR portion of iSH2 (p85-iSH2Δonc, FIG. 3A) synergizes with Ras or Rac1 to activate the PI3K pathway. We transfected NIH3T3 cells were transfected with FLAG-Akt, and activated H-Ras G12V or activated Rac1 Q61L in combination with p85-iSH2Δonc. The Akt kinase activity was measured 48 hours later, following serum starvation for 16 hours. As shown here, the iSH2Δonc, indeed, activates the PI3K/Akt pathway, alone and in synergy with H-Ras or Rac1 (FIG. 3B). The functional interaction between iSH2Δonc and these molecules is likely to be indirect. To show whether the IKR motif is necessary for iSH2Δonc function the intact nSH2/iSH2Δonc and nSH2/iSH2Δonc molecules with carboxy terminal deletions of the IKR or the IKR plus TIF motifs were fused to Hemagglutinin-tagged greenfluorescence protein (HA-GFP). The GFP-fusion constructs were transiently transfected in combination with FLAG-Akt into NIH3T3 cells. Probing an immunoblot of cell lysates with an antibody that recognizes the phosphorylated Thr308 motif of Akt revealed that the deletion of IKR abolishes iSH2Δonc function (FIG. 3C). Thus, the IKR motif is a required component of the GRD domain.

### Example 4

### The LED and cSH2 Motifs of p85 Define a Modular Inhibitory Domain

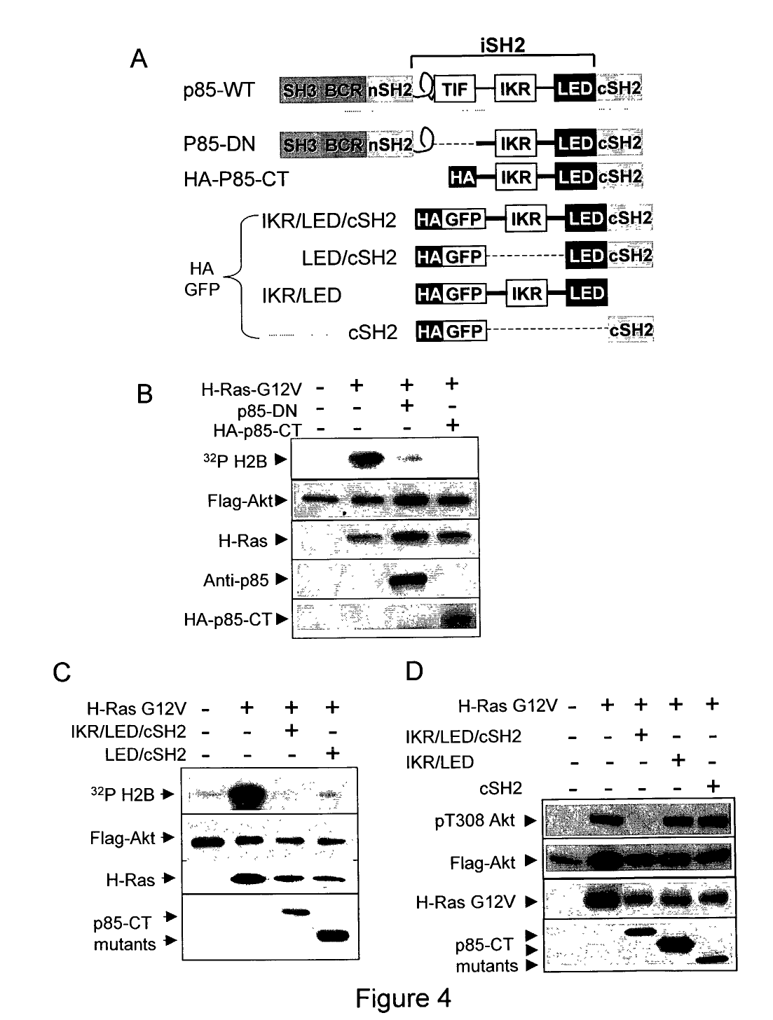

In FIG. 4, it is shown that the carboxy terminal p85 inhibitory domain is modular and consists of the LED and cSH2 motifs. (A) Schematic diagrams of p85 constructs used in the experiments presented here. (B) p85-CT and p85-DN inhibit the activation of the PI3K/Akt pathway by H-Ras-G12V. The indicated constructs in combination with a FLAG-cAkt construct, were transiently transfected into NIH3T3 cells. The Akt kinase activity was measured in anti-FLAG immunoprecipitates. Expression of FLAG-Akt was determined by probing immunoblots of the immunoprecipitates with a rabbit polyclonal anti-Akt antibody. Expression of H-Ras, p85-DN and p85-CT was determined by probing immunoblots of total cell lysates with the corresponding antibodies. (C) The PI3K inhibitory domain consists of the LED and cSH2 motifs. NIH3T3 cells were transfected with the indicated constructs. Akt activity was measured by in vitro phosphorylation of H2B. Expression of the HA-GFP fusion proteins and H-Ras-G12V was determined by probing immunoblots of total lysates with the anti-HA antibody. (D) The cSH2 and IKR/LED motifs alone have no inhibitory activity. NIH3T3 cells were transfected with the indicated constructs. The phosphorylation of Akt at Thr308 was measured by immunoblotting. Expression of the transfected constructs was determined by probing immunoblots with the corresponding antibodies.

Specifically, NIH3T3 cells were transfected with H-Ras G12V in combination with expression constructs of the carboxy terminus of p85 (p85-CT, amino acids 514-725) or p85-DN, a known dominant-negative mutant of p85 (deletion of amino acids 479-513) (Dhand et al., 1994, Embo J 13, 511-21). After overnight serum starvation, the activity of co-transfected FLAG-Akt was examined. The results showed that p85-CT is as efficient as p85-DN at inhibiting the Ras signal (FIG. 4B) and confirmed that the carboxyl terminus of p85 contains a modular inhibitory domain.

To map the p85-CT motifs required for the inhibitory function, p85-CT or portions of it were fused with HA-GFP (FIG. 4A). Expression of the GFP-fusion constructs in combination with H-Ras-G12V in NIH3T3 cells showed that the IKR motif is not required for the p85 inhibitory function (FIG. 4C). Additional deletions showed that the minimal inhibitory domain contains both the LED and the carboxy-terminal SH2 domains. Deletion of either domain abolished inhibitory function (FIG. 4D). Both the inhibitory protein, LED/cSH2, and the noninhibiting cSH2 were able to bind phosphotyrosine targets suggesting that they folded properly (data not shown).

### Example 5

### The p85 Inhibitory Domain Targets PI3K Type IA, and not PTEN

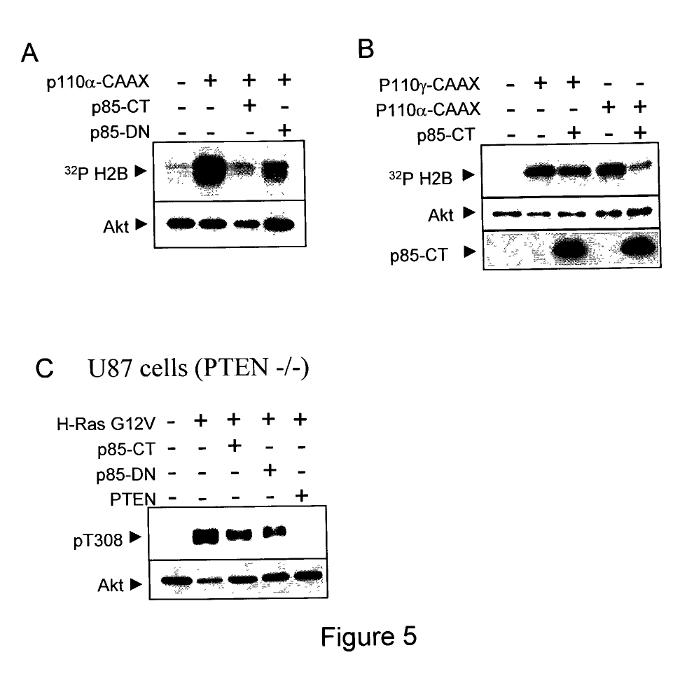

FIG. 5 is presented to show that the PI3K inhibitory domain inhibits p110α, but not p110γ. The inhibition is independent of PTEN. (A, B) p85-CT and p85-DN block Akt activation by constitutively-active p110α-CAAX. NIH3T3 cells were transfected with the indicated constructs. Akt activity was measured by in vitro phosphorylation of H2B. (C) p85-CT and p85DN block Akt activation by Ras in the absence of PTEN. The PTEN-null (Li et al., 1997, Science 275, 1943-7) human glioblastoma cell line, U87MG, was transfected with the indicated constructs. Phosphorylation of Akt at Thr308 was measured in lysates of the transfected cells by immunoblotting.

The p85 inhibitory domain may target molecules that regulate the enzymatic activity of p110. Alternatively, it may target molecules that function upstream of p110 to regulate its activation. To distinguish between these possibilities, we examined whether p85-CT inhibits the activity of p110-CAAX, a membrane bound, constitutive active p110 mutant (Klippel et al., 1996, Molecular & Cellular Biology 16, 4117-27).

Specifically, either p85-CT or p85-DN constructs were transfected into NIH3T3 cells in combination with constitutively-active p110α and FLAG-Akt. In vitro kinase assays of Akt immunoprecipitated from the transfected cells 48 hours later, showed that the p85 inhibitory domain blocks Akt activation by p110α-CAAX (FIG. 5A). Therefore, the p85 inhibitory domain targets molecules that directly regulate the activity of p110. A similar experiment comparing the effects of p85-CT on membrane targeted p110α and p110γ, two type I PI3K molecules of which only the former binds p85, showed that p85-CT inhibits only p110α (FIG. 5B).

PtdIns-3, 4, 5-P₃ levels may be induced because of PI3K activation. Alternatively, they may be induced because of inactivation of PTEN, a tumor suppressor gene that encodes a PtdIns-3, 4, 5-P₃phosphatase (Cantley et al., 1999, Proc Natl Acad Sci USA 96,4240-4245). It is shown here that p85-CT blocks activation of the PI3K/Akt pathway by Ras, even in the absence of PTEN (FIG. 5C) and that the p85 inhibitory domain targets PI3K, and not PTEN. To show that the p85 inhibitory domain targets PI3K and not PTEN, p85-CT or p85-DN constructs were transfected into the PTEN-null cell line, U87MG (Li et al., 1997, Science 275, 1943-7), in combination with constitutively-active H-Ras G12V and FLAG-Akt.

### Example 6

### Tyrosine Kinase Signals Neutralize p85 Inhibitory Function

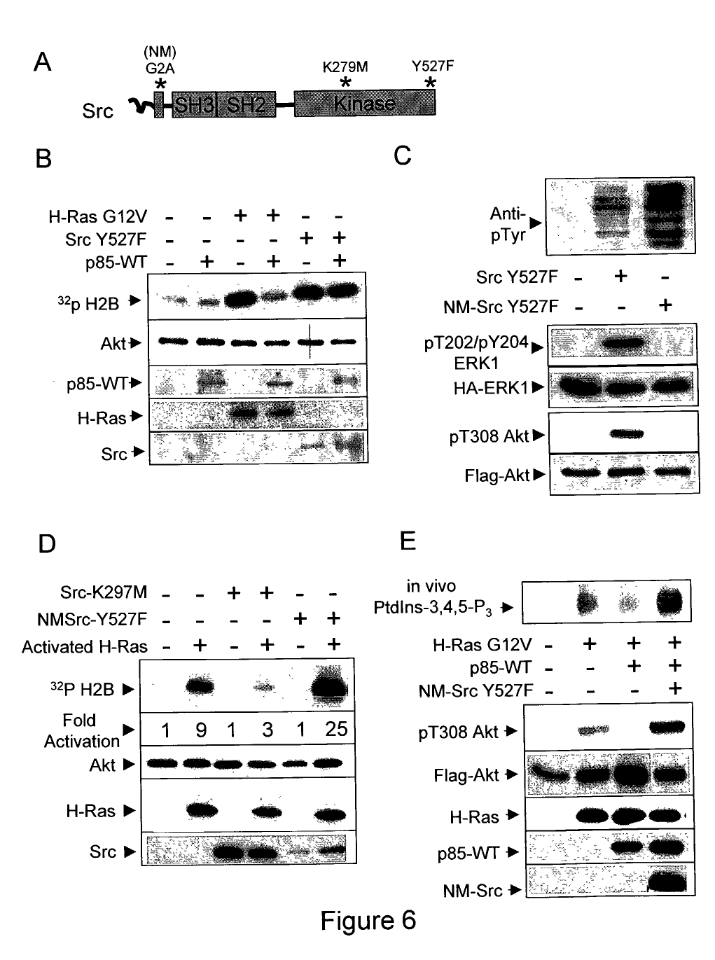

FIG. 6 shows results supporting a finding that Ras-independent Src signals neutralize the p85 inhibitory domain. (A) Schematic diagram of the chicken Src tyrosine kinase and its mutants used in this report. (B) p85-WT inhibits Akt activation by Ras, but not by Src. NIH3T3 cells were transfected with the indicated constructs. Akt activity was measured by in vitro phosphorylation of H2B. The expression of transfected proteins p85-WT, H-Ras and Src-Y527F were monitored by probing immunoblots of total cell lysates with anti-HA antibody (H-Ras and p85-WT) or anti-Src antibody (Src). (C) Mutation of the Src myristoylation signal does not affect Src tyrosine kinase activity, but abolishes Src-induced activation of the Ras/ERK pathway and the PI3K/Akt pathway. The indicated Src constructs were transfected into NIH3T3 cells in combination with either HA-ERK1 or FLAG-Akt. Src tyrosine kinase activity in vivo was determined by probing total cell extracts with the anti-phosphotyrosine antibody, 4G10. HA-ERK1 phosphorylation at Thr202/Tyr204 was measured by probing HA immunoprecipitates with an antibody that recognizes only the phosphorylated ERK1. Phosphorylation of Akt at Thr308 was also measured by immunoblotting. Expression of transfected constructs was determined by probing immunoblots of the cell lysates with the corresponding antibodies. (D) The non-myristoylated Src mutant synergizes with H-Ras to activate the PI3K/Akt pathway. The indicated constructs were transfected into NIH3T3 cells. Akt activity was measured by in vitro phosphorylation of H2B. Expression of transfected constructs was determined by probing immunoblots of total cell lysates with the corresponding antibodies. (E) The non-myristoylated Src abolishes the inhibition of PtdIns-3, 4, 5-P₃ production by p85. Constitutively-active H-Ras-G12V was transfected in combination with p85-WT or non-myristoylated Src Y527F into NIH3T3 cells. Sixteen hours after transfection, ³²P-labeled PtdIns-3, 4, 5-P₃ was measured as in FIG. 1 (top panel). Akt phosphorylation at Thr308 was measured by immunoblotting in cell lysates from duplicate transfections. Expression of transfected constructs was determined by probing immunoblots with the corresponding antibodies.

To show that p85 does not inhibit PI3K activation by Src, a tyrosine kinase, NIH3T3 cells were transfected with expression constructs of p85-WT, H-Ras G12V, Src Y527F and FLAG-Akt in the combination shown in FIG. 6B. In vitro kinase assays of Akt carried out 48 hours later, after overnight serum-starvation, showed that p85-WT blocks Akt activation by H-Ras, but not Src.

Failure of p85 to block PI3K activation by Src is due to neutralization of the p85 inhibitory domain by Src. Therefore, when co-expressed with constitutively-active Src, wild type p85 may become functionally equivalent to p85Δonc.

A Src mutant defective in Ras activation (Marais et al., 1995, Embo J 14, 3136-45) was also used to show that it is able to neutralize the p85 inhibitory domain. The mutation is A G2A mutation that inactivates the Src myristoylation signal (FIG. 6A) without affecting its tyrosine kinase activity (Kamps et al., 1986, Cell 45, 105-12). The G2A mutant (NM-Src) retained full tyrosine kinase activity, but did not activate the Ras/MAP kinase pathway in NIH3T3 cells (FIG. 6C). In addition, this mutant did not activate the PI3K/Akt pathway (FIG. 6C). This mutant, similar to wild-type Src, was able to phosphorylate both endogenous and exogenous p85 (data not shown).

The NM-Src synergizes with Ras to activate the PI3K/Akt pathway. This was shown by co-expressing non-myristoylated Src (NM-Src Y527F) or inactive Src (Src-K297M) with Ras-G12V in NIH3T3 cells. FIG. 6D shows that NM-Src-Y527F and Ras-G12V synergize to activate Akt in NIH3T3 cells supporting that NM-Src neutralizes the p85 inhibitory domain. Following the transfection of cells with H-Ras-G12V and p85-WT in combination with NM-Src, both the induction of PtdIns-3, 4, 5-P₃ and the phosphorylation of Akt at Thr308 in the transfected cells were measured (FIG. 6E). Both measurements showed that p85-WT does not inhibit PI3K activation by Ras signals in the presence of NM-Src. Thus, Src transmits tyrosine phosphorylation signals that neutralize the inhibitory domain of p85.

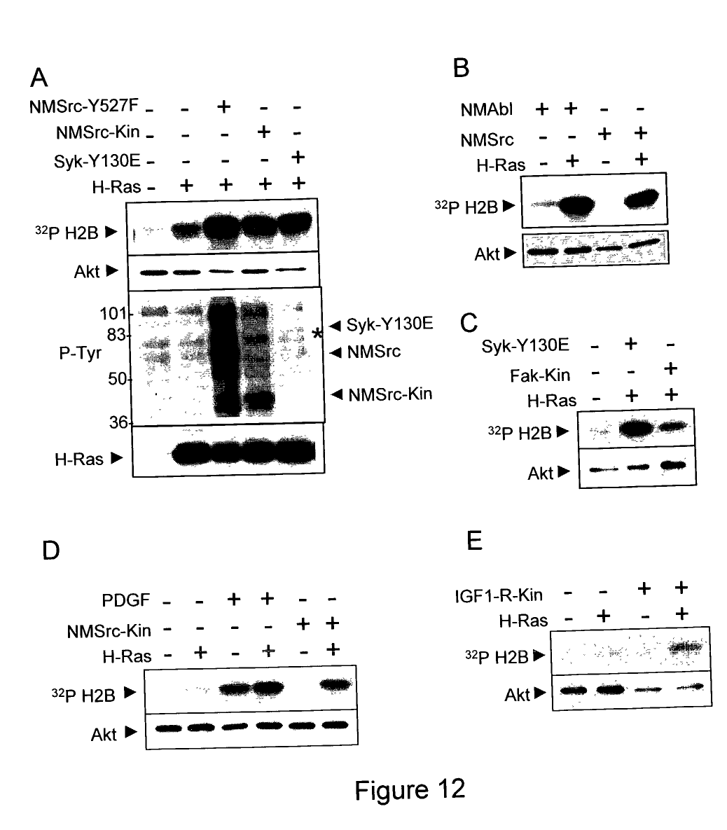

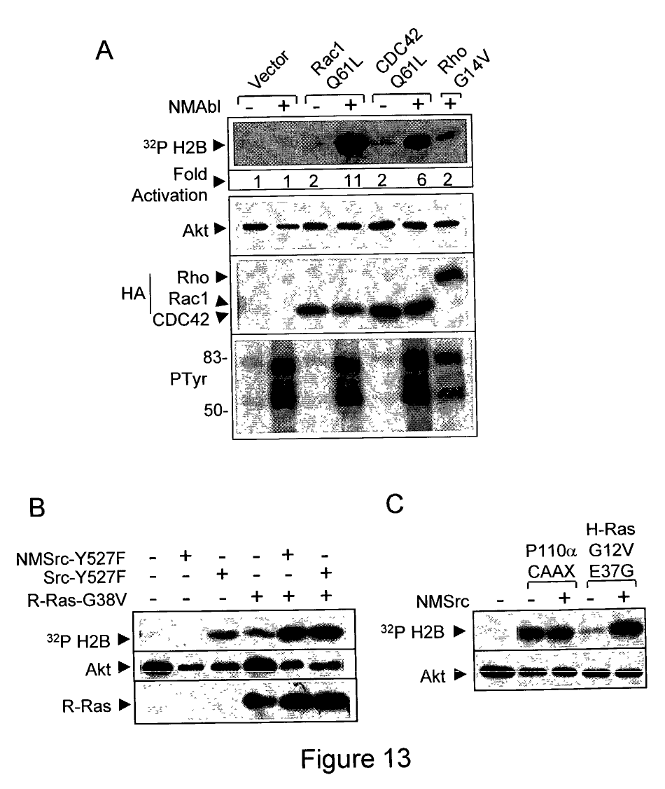

Many cytoplasmic tyrosine kinases and receptor tyrosine kinases are known to activate the PI3K/Akt pathway. Some of these tyrosine kinases for their ability to neutralize the p85 inhibitory domain is shown here. Using synergy with Ras as the assay, it was found that the Src catalytic domain (without its SH2/SH3 domains), other cytoplasmic tyrosine kinases (Syk and Abl, but not p125^{FAK}) and receptor tyrosine kinases (EGF, PDGF and IGF-1 receptors) all neutralize the inhibitory function of p85 (see FIG. 12). Using the same assay, it is also shown here that in addition to Ras and Rac1, small GTPases R-Ras and CDC42, but not RhoA, cooperate with tyrosine kinases to activate the PI3K/Akt pathway (see FIG. 13).

### Example 7

### cSH2 Mutations Abolish p85 Inhibitory Function

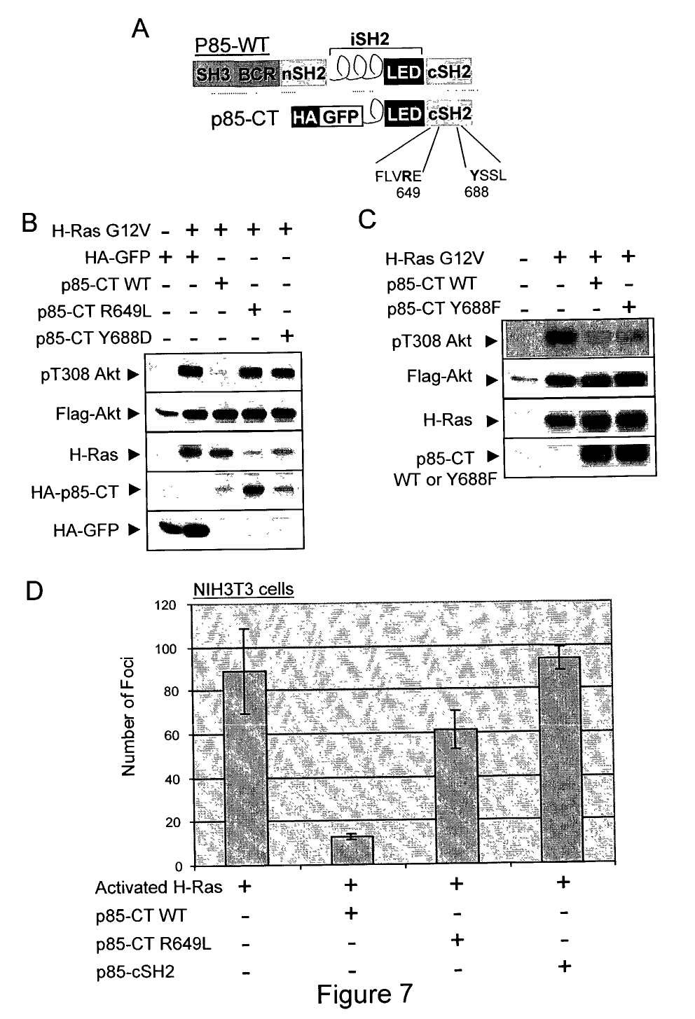

The preceding data show that Src and other tyrosine kinases transmit signals that neutralize the p85 inhibitory domain. In FIG. 7, it is shown that mutations of the phosphotyrosine binding site and the Tyr688 phosphorylation site of the cSH2 motif neutralize the inhibitory domain. (A) Schematic diagram of p85-CT. The diagram shows the phosphotyrosine binding FLVR motif and the Y668 tyrosine phosphorylation site. (B) The R649L and Y688D mutants of p85-CT do not inhibit activation of the PI3K/Akt pathway by Ras. The indicated constructs were transfected into NIH3T3 cells. Akt phosphorylation at Thr308 was determined by immunoblotting. Expression of transfected constructs was determined by probing immunoblots of total cell lysates with the corresponding antibodies. (C) The phosphorylation site mutant Y688F continues to inhibit PI3K/Akt activation by Ras. Expression constructs of p85-CT or p85-CT Y688F were transfected into NIH3T3 cells in combination with FLAG-Akt as indicated. Akt phosphorylation at Thr308 and expression of the transfected constructs were measured by immunoblotting as in B. (D) p85-CT blocks cell transformation by activated H-Ras. NIH3T3 (˜1.5×10⁵) cells, cultured in 100-mm petri dishes, were transfected with expression constructs of constitutively active H-Ras and wild type or mutant p85-CT in the indicated combinations. Three weeks after transfection, the cells were fixed with 70% methanol and they were stained with Giemsa (Chan et al., 1994, Proc Natl Acad Sci USA 91, 7558-62).

The inhibitory domain encompasses the p85 carboxy terminal SH2 motif (cSH2)(FIG. 7A). The p85-CT R649L FLVR mutant, which no longer binds tyrosine phosphorylated molecules (data not shown), was co-expressed in combination with Ras G12V into NIH3T3 cells to show whether the mutant continues to inhibit PI3K/Akt activation by Ras. As shown, the R649L mutation abolished the inhibitory function of p85-CT (FIG. 7B). Thus, phosphotyrosine binding is required for inhibition.

In addition to enabling phosphotyrosine binding, tyrosine kinases, such as Abl and Lck, directly phosphorylate p85 cSH2 at tyrosine residue 688 (von Willebrand et al., 1998, J Biol Chem 273, 3994-4000). To determine whether phosphorylation of this residue is involved in the neutralization of the p85 inhibitory domain, In the present invention, tyrosine residue 688 was mulated to a phosphomimic aspartic acid (p85-CT Y688D). Co-expression of p85-CT Y688D and Ras G12V in NIH3T3 cells showed that this mutation abolished the inhibitory effect of p85CT (FIG. 7B). On the other hand, p85-CT with a tyrosine to phenylalanine mutation at this site (Y688F), continued to block Akt activation by Ras (FIG. 7C). Both p85-CT mutants were able to bind tyrosine phosphorylated molecules (data not shown) indicating that they fold properly. Thus, it is shown here that both phosphotyrosine binding and Y688 phosphorylation regulate p85 inhibitory function.

To show that the inhibitory domain alone also blocks cell transformation the p85-CT together with activated Ras were transfected into NIH3T3 cells. Scoring for foci of transformation two weeks later support that the p85 inhibitory domain is sufficient to block Ras-induced transformation (FIG. 7D). This blockage is specific because both the p85-CT (R649L) and p85-cSH2 constructs failed to block. Blockage of transformation, therefore, correlates perfectly with the blockage of PI3K/Akt activation (FIG. 4D and FIG. 7B).

### Example 8

### Role of Complementary Tyrosine Kinase and Small GTPase Signals on PI3K Activation in Integrin or Growth Factor-stimulated Cells

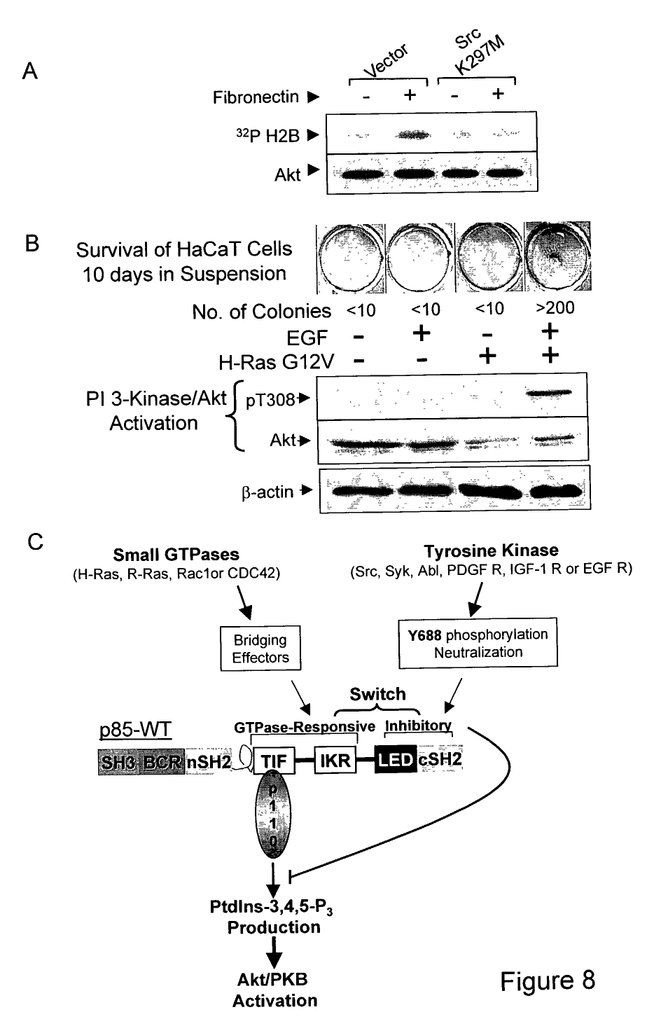

FIG. 8 is presented to complementary roles of tyrosine kinase and small GTPase signals induced by integrin or growth factor stimulation, in PI3K/Akt activation and cell survival. (A) Inhibition of fibronectin-stimulated Akt activity by kinase-inactive Src. Serum starved Mouse 3Y1 fibroblasts, co-transfected with FLAG-Akt and kinase-inactive Sre-K297M or vector, were trypsinized and replated in fibronectin or polylysine-coated plates as indicated. Akt kinase activity in cell lysates harvested 30 minutes later was measured by in vitro phosphorylation of H2B. (B) Activated Ras and EGF cooperate to support long-term cell survival (10 days) and PI3K/Akt activation in HaCaT keratinocytes in suspension cultures. HaCaT cells or HaCaT cells stably-expressing activated H-Ras G12V were cultured on 0.9% agarose in MCDB base medium. EGF was used at 2 nM concentration in the indicated culltures. Cell aliquots were removed after 6 hours in suspension for immunoblot analysis. Cell viability was examined after 10 days in suspension as described (Jost et al., 2001, Mol Biol Cell 12, 1519-27). (C) A model of PI3K activation by tyrosine kinase- and small GTPase signals. According to this model, p85 binds, via its cSH2 motif, tyrosine phosphorylated proteins and undergoes phosphorylation at Y688. Phosphorylation at this site, functionally inactivates the p85 inhibitory domain. Neutralization of the inhibitory domain renders p85 responsive to PI3K activation signals that are transduced by small GTPases.

Mouse 3Y1 fibroblasts were transfected with wild-type FLAG-Akt alone or in combination with kinase-inactive Src K279M. Following serum starvation, the cells were trypsinized and replated onto fibronectin-coated plates or control polylysine-coated plates for 30 minutes. The Akt activation in the fibronectin-coated plates is inhibited by Src K279M (FIG. 8A). Thus, it is shown here that Src signals are required, in combination with small GTPase signals, for PI3K/Akt activation by integrin stimulation.

Suspension cultures of immortalized keratinocytes (HaCaT cells) (Frisch et al., 1994, J Cell Biol 124, 619-26) were also performed to demonstrate the physiological role of complimentary signals transduced by tyrosine kinases and small GTPases. These experiments were carried out in serum-free define media. The constitutively-active H-Ras G12V activates the PI3K/Akt pathway and rescues cells from anoikis only in suspension cultures supplemented with epidermal growth factor (EGF) (FIG. 8B). Ras G12V-alone in the absence of EGF, and EGF-alone in the absence of H-Ras G12V were inefficient in PI3K/Akt activation and failed to rescue the cells from anoikis. Thus, PI3K/Akt activation and inhibition of anoikis depend on the combination of tyrosine kinase and small GTPase signals.

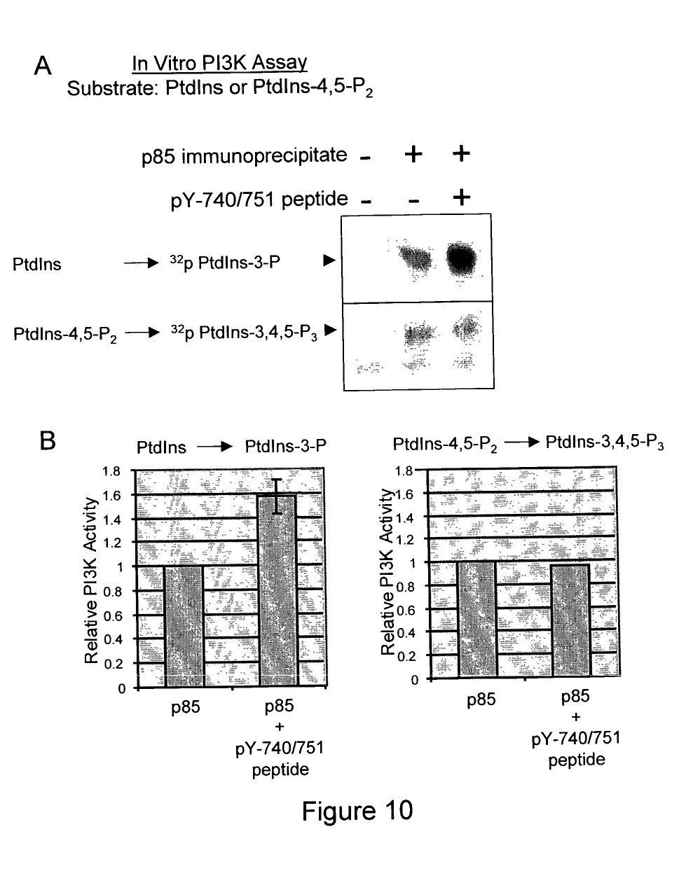

FIG. 9 is presented to demonstrate that Activation of the PI3K/Akt pathway by Ras and Rac1 does not depend on the direct interaction between p110 of PI3K and these molecules. (A) Myrp85Δonc activates the PI3K/Akt pathway in synergy with H-Ras-G12V/E37G, a mutant that does not bind p110. Expression constructs of Myrp85Δonc and FLAG-c-Akt were transfected alone or in combination with HA-tagged H-Ras-G12V, H-Ras-G12V/E37G, and H-RasG12V/Y40C into NIH3T3 cells. Akt activation was determined by measuring the phosphorylation of histone 2B (H2B) by immunoprecipitated Akt. H2B phosphorylation quantitated by phosphorimager is presented in arbitrary units. Expression of FLAG-Akt was determined by probing immunoblots of the immunoprecipitates with a rabbit polyclonal anti-Akt antibody. Expression of H-Ras and p85 constructs was determined by probing immunoblots of total cell lysates with the indicated antibodies. (B) Ras-binding-domain (RBD) of p110 does not inhibit H-Ras-G12V-induced Akt activation. Expression constructs of H-Ras G12V and FLAG-c-Akt were transfected alone or in combination with expression constructs p85-DN and p110β-RBD into NIH3T3 cells. Akt activity was measured and quantitated as illustrated in FIG. 10A. (C) BCR domain of p85 does not inhibit Rac1 Q61L-induced Akt activation. Expression constructs of the Rac1-Q61 L and FLAG-c-Akt were transfected alone or in combination with expression constructs of p85-DN or p85-BCR domain into NIH3T3 cells. Akt activity was measured and quantitated as described in FIG. 10A.

FIG. 10 is presented to show that Phosphotyrosine peptides enhance PtdIns-3-P, not PtdIns-3,4,5-P₃ synthesis by p85 immunoprecipitates in vitro. (A) NIH3T3 cells were serum-starved overnight. Endogenous type IA PI3K was immunoprecipitated using an anti-p85 antibody. After washing, the immunoprecipitates were divided in equal aliquots for in vitro PI3K assays. The substrates used for the assays were either PtdIns or PtdIns-4, 5-P₂. The assays were carried out in the presence or absence of phosphotyrosine peptides that correspond to PDGF-receptor dual PI3K binding site (pY740/pY751). The autoradiogram shows the conversion of Ptdlns to ³²P-labeled PtdIns-3-P and the conversion of PtdIns-4, 5-P₂ to ³²P-labeled PtdIns-3, 4, 5-P₃. (B) The synthesized ³²P-labeled PtdIns-3-P and PtdIns-3, 4, 5-P₃ were measured by Phosphorimager. The graphs shows the average of two independent experiments.

In FIG. 11, it is shown that Ras and p85Δonc do not activate Akt in PDK1 (−/−) embryonic stem cells. Constitutively-active H-RasG12V was co-transfected with p85Δonc and FLAG-c-Akt in PDK1-null embryonic stem cells (Williams et al., 2000, Curr Biol 10, 439-48). Sixteen hours after transfection, the cells were cultured in serum-free media for 5 hours. Phosphorylation of Akt was determined by probing immunoblots of cell lysates with an antibody that recognizes Akt phosphorylated at Thr308. Expression of transfected constructs was determined by probing immunoblots with the corresponding antibodies.

FIG. 12 is presented to show that the Src catalytic domain and other tyrosine kinases, Syk, Abl, PDGF-R and IGF-1-R, but not Fak, synergize with Ras to activate the PI3K/Akt pathway. (A) The Src catalytic domain, NMSrc-Kin, and Syk-Y130E synergize with Ras to activate Akt. (B) Non-myristylated Abl (NMAbl) synergizes with Ras. (C) The catalytic domain of Fak (Fak-Kin) does not synergize with Ras. (D) Stimulation of the PDGF receptor synergizes with Ras. (E) The catalytic domain of the IGF-1 receptor (IGF1-R-Kin) synergizes with Ras. The above tyrosine kinase constructs were co-transfected with H-Ras-G12V/E37G and FLAG-Akt into NIH3T3 cells. Akt activity was measured in vitro using Histone 2B (H2B) as the substrate. Expression of Akt was monitored by probing immunoblots of the Akt immunoprecipitates with a rabbit polyclonal anti-Akt antibody.

FIG. 13 is presented to show that Src and Abl synergize with small GTPases (H-Ras, R-Ras, Rac1 and CDC42), but not with p110, to activate the PI3K/Akt pathway. (A) Rac1 and CDC42, but not RhoA, synergize with non-myristoylated Abl tyrosine kinase in Akt activation. Expression vectors of HA-tagged Rac1-Q61 L, CDC42-Q61L and Rho-G14V, were cotransfected with non-myristylated Abl(NM) and FLAG-Akt into NIH3T3 cells. Akt activity was measured and quantitated as described in FIG. 10A. (B) Src synergizes with R-Ras to activate Akt. Activated R-Ras(G38V) was co-transfected with Src-Y527F or NMSrc-Y527F. Akt activity was measured and quantitated as in FIG. 10A. The expression of transfected constructs was determined by probing Western blots of total cell lysates with the indicated antibodies. (C) Non-myristylated Src does not synergize with P110α-CAAX. NIH3T3 cells were transfected with NMSrc and constitutively active P110α-CAAX or H-Ras(G12V/E37G). Akt activity was measured and quantitated as described in FIG. 10A. The results suggest that p110 is not the target of NMSrc.

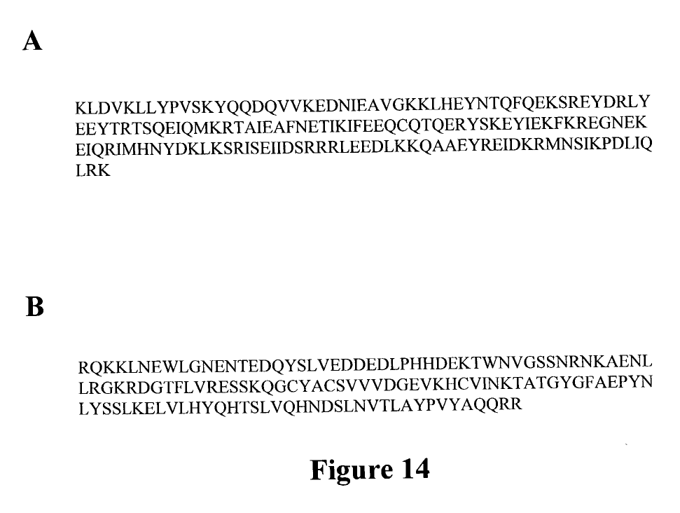

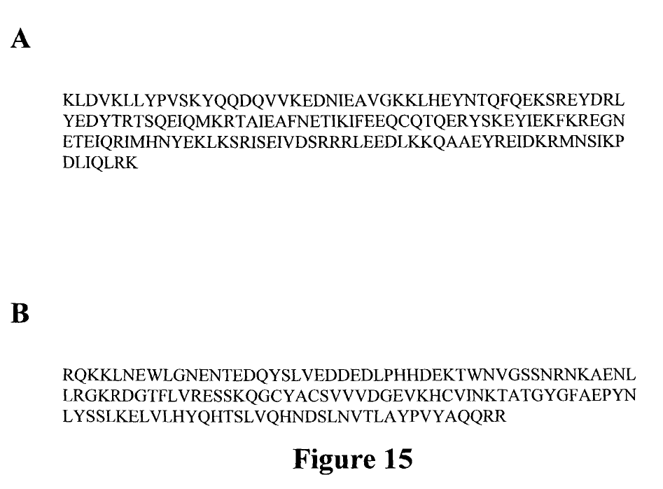

Partial amino acid sequence of p85 alpha subunit is presented in FIGS. 14 and 15. FIG. 14 shows amino acid sequence of human p85 alpha (A) GTPase-Responsive Domain (B) Inhibitory Domain. The GeneBank Accession Number of the full sequence is M61906. The GTPase Responsive Domain amino acid sequence shown herein are amino acids 420 to 575 in the GenBank sequence. The Inhibitory Domain amino acids correspond to amino acids 590 to 724 of the full sequence. FIG. 15 shows amino acid sequence of bovine p85 alpha (A) GTPase Response Domain (B) Inhibitory Domain. The GeneBank Accession Number of the full sequence is M61745. The GTPase Responsive Domain amino acid sequence shown herein are amino acids 420 to 575 in the GenBank sequence and the Inhibitory Domain amino acids correspond to amino acids 590 to 724 of the full sequence.

A description of the vectors, cells, assays and other experimental procedures applicable to one or more of the above Examples is as follows:

NIH3T3 fibrolast cells were cultured in Dulbecco modified Eagle's essential medium (DMEM) supplemented with 10% calf serum and 100 unit/ml penicillin, 100 μg/ml streptomycin and 100 μg/ml kanamycin. Immortalized, non-tumorigenic human keratinocytes (HaCaT cells) (Boukamp et al., 1988, J Cell Biol 106, 761-71) and their tumorigenic variant expressing the activated H-Ras G12V (Boukamp et al., 1990, Cancer Res 50, 2840-7) were used. Transfections were carried out on 1.5-3×10⁵ cells seeded onto 35 mm (6 well) dishes. Cells were transfected using LipofectAMINE (GIBCO-BRL) according to the manufacturer's protocols. Prior to harvesting, cells were cultured overnight in serum-free DMEM medium. Transfection efficiency was monitored by co-transfection of a GFP expression construct. All constructs made in the course of this work were generated in the mammalian expression vector CMV5, using standard molecular biology strategies and were confirmed by DNA sequencing in the core facilities of Kimmel Cancer Center. The Src myristylation signal (MGSSKSKPK), an extended Hemagglutinin epitope (HA) (MASSYPYDVPDYASLGGPSRST), and a FLAG-epitope (MDYKDDDDK) were fused at the amino-terminus of the indicated constructs. Other plasmids used were: Green-fluorescence protein (GFP) expression vector (pFred143(KH1035)), p110-α-CAAX in the pMT2 vector, p110-γ-CAAX in pCDNA3 and H-Ras-G12R-CMV5. These vectors are known to those skilled in this art.

PI3K activity was measured in vitro using endogenous PI3K immunopurified from NIH3T3 mouse fibroblasts (Chan et al., 1990, Mol Cell Biol 10, 3280-3; Layton et al., 1998, J Biol Chem 273, 33379-85). NIH3T3 cells were serum-starved overnight and then lysed with NP40 lysis buffer (30 mM Tris-HCI pH 7.6, 137 mM NaCl, 4% glycerol, 1% NP40, 50 mM NaF, 2.5 mM Sodium pyrophosphate, 5 μg/ml leupeptin, 5 μg/ml aprotinin, 2 mM EDTA, 0.25 mM EGTA, 10 mM PMSF, 1 mM NaVO4 and 2 mM DTT). Endogenous type IA PI3K was immunoprecipitated from these lysates using a rabbit antibody against the PI3K regulatory subunit (p85) (Upstate Biotechnology). The immunoprecipitates were washed sequentially, with lysis buffer, phosphate buffered saline-1% NP40, phosphate buffered saline, 0.5M LiCl-0.1 mM Tris-HCI (pH 7.6), 100 mM NaCl-0.5 mM EGTA-20 mM Tris-HCl (pH 7.6). After washing, the immunoprecipitates were aliquoted to be assayed under different conditions. Some aliquots were incubated for 20 minutes prior to the assay with 12.5 mM of a diphosphotyrosine peptide that corresponds to the target of p85 on the PDGFβ receptor (pY740/pY751) (Layton et al., 1998, J Biol Chem 273, 33379-85). PI3K activity was assayed in 20 mM Tris-HCl pH 7.6, 100 mM NaCl, 0.5 mM EGTA, 10 mM MgCl₂, 10 uCi γ-³²P ATP, 100 uM ATP, 0.1 mg/ml phosphatidylserine (Avanti) using either 0.2 mg/ml PtdIns or 0.2 mg/ml PtdIns-4, 5-P₂ as substrates. Following incubation for 15 minutes at 30° C., phospholipids were extracted with 100 ul of 1M HCl and 300 ul of chloroform/methanol (1:1) and dried under nitrogen gas. Dried phospholipids were dissolved in 30 ul methanol:chloroform (1:4), spotted onto thin-layer chromatography plates (Silica Gel 60, EM Science) and separated in a solvent mixture composed of acetic acid-water-methanol-acetone-chloroform (20:20:50:20:70). ³²P labeled PtdIns-3-P and PtdIns-3, 4, 5-P₃ were visualized by autoradiography and quantitated by Phosphorimager (Molecular Dynamics).

PtdIns-3, 4, 5-P₃ was measured in lipid extracts of ³²P-labeled NIH3T3 cells by thin layer chromatography (Maehama et al., 1998, J Biol Chem 273, 13375-8). Approximately 2×10⁵ NIH3T3 cells plated into 35 mm Petri dishes were transfected with constitutively-active H-Ras G12V, p85-WT and p85Δonc constructs in CMV5 using Fugene 6 (Roche). Sixteen hours later, the transfected cells were serum starved for 5 hours, and following this, they were labeled for 1.5 hours with 100 uci/ml ³²P-orthophosphate in a phosphate-free buffer (10 mM Hepes pH 7.5, 136 mM NaCl, 4.9 mM KCl and 5.5 mM glucose). For wortmannin treatment, 200 nM wortmannin was added to cells 45 minutes prior to cell extraction. Following removal of the labeling buffer, we added 1 ml of 1% HCl:methanol-chloroform (1:1.3:2.6) and 0.5 ml of 8% HCl-chloroform (1:1) were added to the cells. The organic phase was washed once with 1% HCl and then dried under nitrogen gas. Dried lipid extracts were dissolved in 50 ul methanol:chloroform (5:95), spotted onto thin-layer chromatography plates (Silica Gel 60, EM Science) and separated in the following solvent mixture: acetic acid-water-methanol-acetone-chloroform (20:20:50:20:70). ³²P labeled PtdIns-3, 4, 5-P₃ was visualized by autoradiography and quantitated by Phosphorimager (Molecular Dynamics).

Akt/PKB activation by phosphorylation strictly depends on the binding of the Akt PH domain to PtdIns-3,4,5-P₃ and therefore, indirectly measures intracellular PtdIns-3,4,5-P₃ levels (Chan et al., 1999, Ann Rev Biochem 68, 965-1015). Akt/PKB kinase activation was determined either by measuring the phosphorylation of Akt at Thr308 using an antibody from Cell Signaling Technology or by measuring the kinase activity of immunoprecipitated FLAG-tagged wild type Akt. The activity of immunoprecepitated Akt was measured using Histone 2B as the substrate, and following previously described protocols (Bellacosa et al., 1998, Oncogene 17, 313-25; Franke et al., 1995, Cell 81, 727-736) with slight modifications. Briefly, cells were lysed on ice using a NP40 lysis buffer (25 mM Tris-HCl pH 7.6, 137 mM NaCl, 10% glycerol, 1% NP40, 10 mM NaF) freshly supplemented with 1 mM Sodium pyrophosphate, 5 μg/ml leupeptin, 5 μg/ml aprotinin, 1 mM EDTA, 10 mM PMSF, 1 mM NaVO4 and 1 mM DTT. Clarified cellular lysates were incubated for 1.5-2 hr with 5 μl of a 50% suspension of anti-FLAG antibody (M2) crossed-linked to agarose beads (Sigma) and 15 μl of protein G -agarose (GIBCO-BRL). The immunoprecipitates were washed on ice three times with the stock lysis buffer supplemented with 1 mM Sodium pyrophosphate, 1 mM NaVO4 and 1 mM DTT. Subsequently, they were washed once with 50 mM Tris-HCl pH 7.5, and once with 10 mM HEPES pH 7.5, 10 mM MgCl₂ and 1 mM DTT. Kinase assays were carried out on washed immunoprecipitates and it was initiated by the addition of 20 μl kinase assay mix (10 μCi (γ-³²P)ATP (3000 Ci/mmole, Amersham), 0.1 μg/ml H2B (Boehringer-Mannheim), 5 μM ATP, 10 mM MgCl₂, 10 mM MnCl₂, and 20 mM HEPES, pH 7.5). Following a 15 min incubation at 25° C., reactions were stopped by the addition of SDS-polyacrylamide gel electrophoresis (SDS-PAGE) sample buffer. The samples were boiled and, following electrophoresis in a 13% SDS-PAGE, they were transferred onto PVDF membranes (Immobilon P, Millipore) with an X-genie electro-blotter apparatus (Ideas Scientific). The phosphorylated Histone H2B was visualized by autoradiography and quantitated by Phosphorlmager (Molecular Dynamics). Akt expression was determined by probing the membranes with a polyclonal rabbit anti-Akt antibody.

Specific Akt kinase activity was determined by Phosphorimager-quantitation of the ³²P-phosphorylated Akt substrate Histone 2B, and by quantification of Akt in anti-Akt immunoprecipitates using a laser densitometry scanner (Molecular Dynamics). The maximum difference in protein expression between different transfections was less than 50%.

Total cell lysates and protein immunoprecipitates were subjected to SDS-PAGE. The SDS-PAGE-resolved proteins were transferred onto PVDF membranes (Immobilon P, Millipore) and the resulting immunoblots were probed with the appropriate antibodies. The blots were blocked for 10 min with 1% skim milk in Tris-buffered saline containing 0.1% Tween-20 for 30 minl. Antibodies, diluted in the same buffer, were incubated with the blots at room temperature for 2-3 hrs or at 4° C. overnight. Binding of antibodies to membrane-immobilized proteins was visualized by enhanced chemiluminescence (ECL, Amersham) following the manufacturer's protocol.

The blots probed with anti-phosphotyrosine antibody were first blocked with 2% Ficoll, 2% bovine serum albumin, 2% polyvinyl pyrrolidone for 0.5 hour. Monoclonal anti-phosphotyrosine antibodies PY20 (Transduction Lab), 4G10 (Upstate Biotechnology) or PY100 (New England Biolabs) were diluted 1:1000 in Tris-buffered saline containing 0.1% Tween-20 supplemented with 8% fetal calf serum. Diluted antibody was incubated with the blots at room temperature for 1 hour. The blots were processed and visualized by enhanced chemiluminescence (ECL, Amersham).

The following antibodies were used at 1:1000 dilution: The following antibodies were used: Anti-HA mouse monoclonal (HA. 11, Covance), anti-FLAG mouse monoclonal (M2 and M5, Sigma), anti-p85α mouse monoclonal (Transduction Laboratories), anti-p85α rabbit polyclonal (Upstate Biotechnology), anti-Ras mouse monoclonal (Transduction Laboratorie), anti-Akt rabbit polyclonal (Franke et al., 1995, Cell 81, 727-736), anti-Src mouse monoclonal (gift from T. Parsons), anti-phosphoErk (Thr202/Tyr204) rabbit polyclonal (Cell Signaling Tech.), anti-phosphoAkt (Thr308) rabbit polyclonal (Cell Signaling Tech.), anti-phosphotyrosine antibodies (4G10, Upstate Biotechnology or PY100, Cell Signaling Tech.).

Transformation assays in NIH3T3 cells were performed as previously described (Chan et al., 1994, Proc Natl Acad Sci USA 91, 7558-62; Wigler et al., 1977, Cell 11, 223-32). Briefly, approximately 1.5×10⁵ NIH3T3 cells were plated onto a 100-mm culture dish. Activated H-Ras G12R and p85-derived constructs in Elongation Factor promoter-based vector, pCEFL KZ AU5 (Crespo et al., 1997, Nature 385, 169-72) were transfected by standard calcium phosphate precipitation method in triplicate. After transfection, cell culture media(DMEM supplemented with 5% calf serum) were changed twice a week and the number of foci were scored weekly for 3 weeks. Cells were then fixed in 70% methanol and stained with Giemsa for counting.

Suspension survival assay were performed as previously described (Jost et al., 2001, Mol Biol Cell 12, 1519-27). Suspension cultures of HaCaT cells (Boukamp et al., 1988, J Cell Biol 106, 761-71) and their tumorigenic variant expressing the activated H-Ras G12V (Boukamp et al., 1990, Cancer Res 50, 2840-7) were initiated by seeding cells on top of 0.9% agarose gels using MCDB153 base medium supplemented with 0.2% (vol/vol) fatty-acid-free bovine serum albumin and free of protein growth factors unless stated otherwise. Cell aliquots were harvested after 6 hours in suspension for protein exptraction and immunoblot analysis. Cell viability was measured after 10 days in suspension. Cell viability was analysed by crystal violet staining of cells reseeded after 10 days of suspension culture onto tissue culture-treated plastic and allowed to grow for 3 days. EGF was used at 2 nM (10 ng/ml).

All publications and references, including but not limited to patents and patent applications, cited in this specification, are herein incorporated by reference in their entirety as if each individual publication or reference were specifically and individually indicated to be incorporated by reference herein as being fully set forth. While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. A method of modulating phosphoinositide 3-kinase (PI3K) activity or phosphatidylinositol-3,4,5-P₃ (PIP3) production comprising abrogating GTPase-Responsive Domain in p85 subunit of the PI3K.

2. The method of claim 1, wherein the GTPase-Responsive Domain has a sequence set forth in FIG. 14A.

3. The method of claim 2, wherein said abrogation is by means of mutation of sequences in said domain such that small GTPases cannot regulate PI3K activity or PIP3 production by functionally interacting with said domain.

4. The method of claim 2, wherein said abrogation is achieved by way of antisense sequences to said domain or antibodies specific to said domain.

5. A method of modulating phosphoinositide 3-kinase (PI3K) activity or phosphatidylinositol-3,4,5-P₃ (PIP3) production comprising overexpressing only inhibitory domain portion of p85 subunit of PI3K.

6. The method of claim 5, wherein the inhibiotory domain portion has a sequence set forth in FIG. 14B.

7. A method of modulating ras-induced phosphatidylinositol-3,4,5-P₃ (PIP3) synthesis in a mammalian cell, comprising: administering a vector capable of expressing a p85 inhibitory domain comprising amino acids set forth in FIG. 14B to a mammal wherein the vector is targeted to the mammalian cell, wherein the mammalian cell is a cancer cell, wherein the cancer cell has a constitutively active ras oncogene.

8. The method of claim 7, wherein the mammal is a human.

9. The method of claim 8, wherein the cancer cell is that of leukemia, lymphoma, myeloma, a glioblastoma, melanoma, breast cancer, prostate cancer or lung cancer.

10. The method of claim 7, wherein the vector is an adenoviral vector.

11. A method for selecting modulators of phosphatidylinositol-3,4,5-P₃ (PIP3) induction by a small GTPase, comprising:
providing a first host cell of a given type transfected with a construct expressing GTPase responsive domain of p85 subunit of phosphoinositide 3-kinase (PI3K) wherein the first host cell expresses the small GTPase which GTPase has functional interaction with the GTPase responsive domain;
providing a seond host cell of the same type not transfected with said construct;
contacting the first and second host cells with a test compound; and
analyzing PIP3 levels in the first and the second host cells and identifying the test compound capable of modulating PIP3 induction by the small GTPase.

12. The method of claim 11, wherein the host cell is a cancer cell, a tumorigenic cell or a yeast cell.

13. The method of claim 11, wherein the test compound is an agonist or an antagonist.

14. The method of claim 13, wherein the agonist or an antagonist comprises a peptide, a small molecule or an organic molecule.

15. The method of claim 11, wherein the antagonist binds to a binding site of the GTPase responsive domain thereby preventing the functional interaction between the small GTPase and GTPase responsive domain.

16. The method of claim 15, wherein the small GTPase is ras.

17. The method of claim 11, wherein the agonist binds to a binding site of the GTPase responsive domain or the the small GTPase, thereby promoting the functional interaction between the small GTPase and GTPase responsive domain.

18. The method of claim 11, wherein said GTPase responsive domain comprises an amino acid sequence set forth in FIG. 14.

19. A method for selecting modulators of phosphatidylinositol-3,4,5-P₃ (PIP3) induction by a small GTPase, comprising:
expressing, in the presence of a test compound, GTPase responsive domain of p85 subunit of phosphoinositide 3-kinase (PI3K) in a first host cell of a given type transfected with a construct capable of expressing said domain wherein the first host cell expresses the small GTPase capable of functionally interacting with said domain;
providing a seond host cell of the same type not transfected with the construct but exposed to the test compound, wherein the second host cell expresses the small GTPase capable of functionally interacting with said domain; and
analyzing PIP3 levels in the first and the second host cells and identifying the test compound capable of modulating PIP3 induction by the small GTPase.

20. The method of claim 19, wherein the given type of host cell is a cancer cell, a tumorigenic cell or a yeast cell.

21. A recombinant polynucleotide segment, comprising a polynucleotide fragment of phosphoinositide 3-kinase (PI3K), wherein the polynucleotide fragment encodes a polypeptide with an amino acid sequence set forth in FIG. 14B or FIG. 15B but not a polypeptide with an amino acid sequence in FIG. 14A or FIG. 15A.

22. A vector comprising the recombinant polynucleotide segment of claim 21.

23. The vector of claim 22, wherein the recombinant polynucleotide segment encodes the polypeptide capable of inhibiting p110.

24. A host cell transfected with the vector of claim 22.

25. A recombinant polynucleotide segment, comprising a polynucleotide fragment of phosphoinositide 3-kinase (PI3K), wherein the polynucleotide fragment encodes a polypeptide with an amino acid sequence set forth in FIG. 14B or FIG. 15B except that the polypeptide has a mutation to disable neutralization of the polypeptide by tyrosine kinase transmitted signals.

26. The recombinant polynucleotide segment of claim 25, wherein the mutation is of tyrosine residue at position 99 in the amino acid sequence set forth in FIG. 14B or FIG. 15B.

27. A vector comprising the recombinant polynucleotide segment of claim 25.

28. The vector of claim 27, wherein the recombinant polynucleotide segment encodes the polypeptide capable of inhibiting p110.

29. A host cell transfected with the vector of claim 28.
